(19) United States
(12) Patent Application Publication
(10) Pub. No.: US 2025/0099609 A1
(43) Pub. Date: Mar. 27, 2025

(54) MULTIFUNCTIONAL CERIUM OXIDE-P7C3 (AMINOPROPYL CARBAZOLE) CHIMERIC NANOCOMPOSITIONS AND METHODS FOR USE THEREOF

(71) Applicant: University of Central Florida Research Foundation, Inc., Orlando, FL (US)

(72) Inventors: Melanie Coathup, Orlando, FL (US); Sudipta Seal, Orlando, FL (US); Fei Wei, Orlando, FL (US); Elayaraja Kolanthai, Orlando, FL (US)

(73) Assignee: University of Central Florida Research Foundation, Inc., Orlando, FL (US)

(21) Appl. No.: 18/892,448

(22) Filed: Sep. 22, 2024

Related U.S. Application Data
(60) Provisional application No. 63/539,843, filed on Sep. 22, 2023.

(51) Int. Cl. A61K 47/69; A61K 47/55; A61P 19/10; A61P 43/00

(52) U.S. Cl. CPC A61K 47/6929; A61K 47/55; A61P 19/10; A61P 43/00

(57) ABSTRACT

Multifunctional cerium oxide-P7C3 (aminopropyl carbazole) chimeric nanocompositions and methods for using these nanocompositions for enhancing bone deposition are provided. For example, these nanocompositions can be used to combat radiation-induced bone tissue/cell damage, to treat any disorders that promote osteoporosis, and/or to treat bone trauma/injury.

### CROSS REFERENCE TO RELATED APPLICATIONS

This application claims the benefit of U.S. provisional patent application Ser. No. 63/539,843, filed Sep. 22, 2023, which is incorporated herein by reference.

### FIELD OF THE INVENTION

The invention generally relates to analysis of the mechanisms of bone loss and bone deposition; particularly to medical strategies for enhancing bone deposition; and most particularly to multifunctional cerium oxide-P7C3 (aminopropyl carbazole) chimeric nanocompositions for enhancing bone deposition to combat radiation-induced bone tissue/cell damage, to treat any disorders that promote osteoporosis, and/or to treat bone trauma/injury.

### BACKGROUND

Cancer remains a leading cause of death worldwide. A combination of surgical resection, radiotherapy, and chemotherapy is commonly used in the treatment of various local and metastasizing cancer cells¹. One-half to nearly two-thirds of cancer patients will be exposed to controlled, radiotherapeutic levels of ionizing radiation (IR) at some point during their care²⁻⁴. Although IR is a fundamental and necessary tool, exposure of adjacent noncancerous tissues to IR is inevitable and can lead to major morphological and functional damage to otherwise healthy tissue. Due to its high calcium content, bone is estimated to absorb 30%-40% more IR than other tissues, making it a common site for serious ancillary tissue damage⁵. Bone marrow failure, immunosuppression, osteoradionecrosis, osteoporosis, pathologic insufficiency fractures, and subsequent fracture non-unions are significant complications associated with radiotherapy in cancer survivors, even with fractionation of treatments⁶⁻¹¹. To date, no effective therapy exists. As such, the burden of IR-induced damage to healthy bone is a persistent and substantial source of functional impairment, pain, disability, and morbidity^{5,7,9,10,12-14}.

Treatment with P7C3 (Pool 7-Compound 3, aminopropyl carbazole) has been shown to be an effective countermeasure against ionizing radiation-induced bone damage and/or bone loss¹⁵. P7C3 is an antiapoptotic aminopropyl carbazole with high oral bioavailability that exerts its activity through activation of the intracellular enzyme nicotinamide phosphoribosyltransferase (NAMPT). NAMPT is an adipokine known to be the rate-limiting enzyme in the NAD⁺ salvage pathway and directly increases NAD⁺ levels and NAD⁺-dependent enzyme activity^{16,17}. Mechanistically, P7C3 increases NAD flux in mammalian cells and can restore cell function under conditions of overwhelming energy crisis that would normally lead to cell death^{16,18.}

Treatment with cerium oxide nanozymes has also been shown to be an effective countermeasure against ionizing radiation-induced bone damage and/or bone loss¹⁹. At reactive surface sites, cerium ions have the ability to easily undergo redox recycling and thus, are capable of drastically adjusting their electronic configurations and versatile catalytic activities. Wei et al. have shown that an engineered artificial nanozyme composed of cerium oxide and designed to possess a higher fraction of trivalent (Ce³⁺) surface sites (relative to Ce⁴⁺), mitigates ionizing radiation-induced loss in bone area, bone architecture, and strength¹⁹.

Although these various treatments are encouraging, a more effective remedy to counteract bone tissue damage and improve quality of life for patients is urgently needed.

### SUMMARY OF THE INVENTION

The invention described herein provides this urgently needed more effective remedy. The experiments described show that the administration of a cerium oxide nanoparticle (CNPs) formulation, with increased levels of Ce³⁺ surface sites relative to Ce⁴⁺, reduces radiation-induced bone tissue, organ, and peripheral blood cell damage. Further, and via a differing mechanism, the small molecule P7C3 (aminopropyl carbazole), also reduces tissue damage. At therapeutic levels, the small molecule P7C3 significantly reduces primary and metastatic cancer cell growth. This invention combines these two unique approaches as a bi-modal and multifunctional chimeric nanostrategy to combat against ionizing radiation-induced damage. As the cerium oxide nanoparticles and P7C3 work via different mechanisms, a synergistic effect will improve on either individual effect, providing a first of a kind strategy to combat against radiation-induced tissue/cell damage and disorders that promote osteoporosis.

The invention described herein holds the promise of being safe, tolerable, and capable of delivering stable and persistent activity. Synthesis (of the nanocompositions) is suitable for scale up manufacturing in preparation for clinical application. The inventive nanocompositions and methods can be used in a wide variety of situations; for example, but not limited to, for prevention of radiation-induced damage in astronauts, for patients undergoing radiotherapy, for individuals exposed to chronic low dose (e.g., nearby nuclear power stations) radiation, individuals exposed to acute high dose levels of radiation due to accidental or terroristic nuclear events, for patients having bone trauma/injury, and for patients who may develop osteoporosis for any reason (non-limiting e.g. post-menopause, due to aging, or bone loss associated with extensive bed rest or immobility, or diabetes).

The terms “P7C3”, “aminopropyl carbazole” and “Pool 7, Compound 3” are used interchangeably herein. The terms “cerium oxide nanoparticles”, “CNPs”, “64C”, and “CeONPs” are used interchangeably herein. The term “64C-73” refers to the inventive nanocomposition including both P7C3 (aminopropyl carbazole) and cerium oxide nanoparticles (CNPs). The terms “radiotherapy” and “radiation therapy” are used interchangeably herein.

In a general aspect, the invention provides compositions and methods for protecting bone from damage.

In another general aspect, the invention provides compositions and methods for encouraging bone growth.

In a general aspect, the invention provides a medical strategy for enhancing bone deposition.

In another general aspect, the invention provides a medical strategy for preventing bone damage and bone breakdown.

In another general aspect, the invention provides a medical strategy for decreasing bone loss while simultaneously encouraging bone deposition.

In an aspect, the invention provides compositions and methods that are capable of counteracting bone tissue damage while simultaneously inhibiting cancer growth.

In an aspect, the invention provides a composition comprising P7C3 (aminopropyl carbazole) and cerium oxide nanoparticles (CNPs). A cerium oxide nanoparticle is an artificial enzyme capable of functioning as an antioxidant.

In another aspect, the invention provides a nanocomposition comprising P7C3 (aminopropyl carbazole) attached/conjugated to cerium oxide nanoparticles (CNPs). The cerium oxide nanoparticles have increased levels of Ce³⁺ surface sites relative to levels of Ce⁴⁺ surface sites, such as, but not limited to cerium oxide CeONP^{60/40}. The P7C3 is attached to the cerium oxide nanoparticles via a crosslinker. The type and the length of the crosslinker are essential parameters for the design and biological activity of chimeras. The length of the crosslinker is controlled to avoid steric hinderance of the P7C3 and any unfavorable surface kinetic interactions with the cerium oxide nanoparticles. This will allow optimization of drug release and therapeutic efficacy. Some crosslinkers cleave immediately on contact with body fluid and others will cleave when entering a cell. Any linkers or crosslinkers capable of performing the desired function are contemplated for use. Nonlimiting examples of such linkers or crosslinkers are CD1 (N,N′-carbonyldiimidazole) and polyethyleneimine.

In another aspect, the invention provides a pharmaceutical composition comprising a nanocomposition including P7C3 (aminopropyl carbazole) attached/conjugated to cerium oxide nanoparticles and at least one pharmaceutically acceptable carrier. The phrase “pharmaceutically acceptable carrier” refers to an inactive and non-toxic substance used in association with an active substance, i.e. a nanocomposition including P7C3 (aminopropyl carbazole) attached to cerium oxide nanoparticles, especially for aiding in the application/delivery of the active substance. A non-limiting example is subcutaneous delivery of the nanocomposition as an injection or via IV. “Inactive”, in this context, refers to inactivity with regard to the activity of the active substance. Non-limiting examples of pharmaceutically acceptable carriers are diluents, fillers, binders, disintegrants, superdisintegrants, flavorings, sweeteners, lubricants, alkalizers/alkalinizing agents, and absorption enhancers/penetration enhancers/permeation enhancers. Pharmaceutically acceptable carriers can be in any usable form such as a solid or a liquid. Further, pharmaceutically acceptable carriers can have more than one function, a non-limiting e.g., a filler can also be a disintegrant. Additionally, pharmaceutically acceptable carriers may also be referred to as non-medicinal ingredients (NMIs) or pharmaceutically acceptable excipients. Any pharmaceutically acceptable carrier used for production and/or delivery of drugs is contemplated for use with the described nanocompositions.

The pharmaceutical composition can also include a therapeutically effective amount of the nanocomposition. The nanocomposition can include a therapeutically effective amount of cerium oxide nanoparticles conjugated with/attached to a therapeutically effective amount of P7C3 (aminopropyl carbazole). The phrase “effective amount”, “pharmaceutically effective amount” or “therapeutically effective amount” refers to the amount of a composition necessary to achieve the composition's intended function, for example, in the instant invention, protection of bone and/or enhancement of bone deposition.

In another aspect, the components of the inventive compositions/nanocompositions can be packaged in containers and assembled in kits together with instructions for use. The P7C3 and cerium oxide nanoparticles can be included separately in a kit with a crosslinker or in kit as the linked/conjugated nanocomposition.

In an embodiment, the invention provides various methods for use of the inventive nanocompositions, particularly, but not limited to, methods for enhancing bone deposition in bone tissue and/or in a subject. This method includes non-limiting steps for providing a pharmaceutical composition including a nanocomposition having P7C3 (aminopropyl carbazole) attached to cerium oxide nanoparticles and at least one pharmaceutically acceptable carrier; and administering the pharmaceutical composition to the bone tissue or to the subject. Although preferably a human, the subject is not limited thereto. A “subject” refers to any human or animal that can benefit from the methods and treatments disclosed herein. The subject can be a person or animal exposed to radiation, a patient receiving radiation therapy, a patient that will receive radiation therapy, a patient that has received radiation therapy, a patient having osteoporosis, a patient having a condition that causes osteoporosis, a patient having cancer, or a patient having bone trauma/injury. Osteoporosis refers to any condition that weakens bone and/or renders bone easily breakable. The compositions/nanocompositions and methods described herein can be used as a preventive for and/or as a treatment for osteoporosis.

In another embodiment, the invention provides various methods for use of the inventive nanocompositions, particularly, but not limited to, methods for protecting bone tissue of a subject from radiation damage. These methods include subjects both exposed to radiation (e.g. patients undergoing radiotherapy) and not exposed to radiation but likely to be exposed (e.g. astronauts). This method includes non-limiting steps for providing a pharmaceutical composition including a nanocomposition having P7C3 (aminopropyl carbazole) attached to cerium oxide nanoparticles and at least one pharmaceutically acceptable carrier; and administering the pharmaceutical composition to the subject exposed to or to the subject not exposed to radiation. Non-limiting examples of administration are administration of the pharmaceutical composition to subject prior to radiation exposure and administering the pharmaceutical composition to the subject prior to radiation therapy, concurrently with radiation therapy, or at a predetermined time after radiation therapy. Non-limiting examples of a predetermined time are 24 hours and 48 after radiation exposure/therapy. The compositions/nanocompositions and methods described herein can be used for treating cancer and protecting bone tissue from radiation damage in a subject undergoing radiation therapy for the cancer. Thus, cancer is treated, and bone protected simultaneously.

In an embodiment, the invention provides various methods for use of the inventive nanocompositions, particularly, but not limited to, methods for treating bone trauma/injury in a subject. The nanocompositions can be effective in boosting bone repair following injury or trauma by accelerating repair of the bone tissue (e.g. after fracture). This method includes non-limiting steps for providing a pharmaceutical composition including a nanocomposition having P7C3 (aminopropyl carbazole) attached to cerium oxide nanoparticles and at least one pharmaceutically acceptable carrier; and administering the pharmaceutical composition to the subject.

In another embodiment, the invention provides various methods for use of the inventive nanocompositions, particularly, but not limited to, methods for protecting DNA of bone cells in a subject exposed to radiation. This method includes non-limiting steps for providing a pharmaceutical composition including a nanocomposition having P7C3 (aminopropyl carbazole) attached to cerium oxide nanoparticles and at least one pharmaceutically acceptable carrier; and administering the pharmaceutical composition to the subject. In an alternative embodiment, the bone cells can be treated ex vivo. The inventive nanocomposition (64C-73) and cerium oxide nanoparticles (64C) protect against DNA damage due to irradiation. Non-limiting examples of administration are administration of the pharmaceutical composition to subject prior to radiation exposure and administering the pharmaceutical composition to the subject prior to radiation therapy, concurrently with radiation therapy, or at a predetermined time after radiation therapy. Non-limiting examples of a predetermined time are 24 hours and 48 after radiation exposure/therapy.

In another embodiment, the invention provides various methods for use of the inventive nanocompositions, particularly, but not limited to, methods for reducing gene expression of pro-inflammatory markers in a subject. Radiation exposure increases levels of harmful, pro-inflammatory proteins, such as, but not limited to interleukin-1β (II-1β), interleukin-6 (IL-6), receptor activator of nuclear factor kappa-B ligand (RANKL), and cathepsin-K (CTSK). This method includes non-limiting steps for providing a pharmaceutical composition including a nanocomposition having P7C3 (aminopropyl carbazole) attached to cerium oxide nanoparticles and at least one pharmaceutically acceptable carrier; and administering the pharmaceutical composition to the subject.

The above-listed aspects are exemplary embodiments only and are not meant to limit the invention. Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 shows results of a cell viability analysis. Osteoclasts were treated with P7C3, with cerium oxide nanoparticles (CNPs), or with a combination of P7C3 and CNPs. This treatment was carried out without exposing the osteoclasts to radiation.

FIGS. 2A-B show analysis of osteoclast maturation when the cells were treated with P7C3, with cerium oxide nanoparticles (CNPs), or with a combination of P7C3 and CNPs. This treatment was carried out without exposing the osteoclasts to radiation. FIG. 2A shows staining of the treated osteoclasts with DAPI, Phalloidin, and a merger of DAPI and Phalloidin staining and FIG. 2B shows micrographs, low and high magnification, of the stained osteoclasts.

FIG. 3 shows analysis of stem cell conversion to fat cells after treatment with P7C3, with CeONP^{60/40}, or with CeONP^{20/80}. All treatment was administered with radiation (IR 21d).

FIGS. 4A-B show micrographs of assessed bone mineral formation after treatment with P7C3, with cerium oxide nanoparticles (CNPs), or with a combination of P7C3 and CNPs. FIG. 4A shows micrographs, both low and high magnification, of bone (mineral) formation when treatment was administered 24 hours after exposure to radiation and FIG. 4B shows micrographs, both low and high magnification, of bone formation when treatment was administered 48 hours after exposure to radiation (7 Gy).

FIGS. 5A-B show micrographs of assessed bone formation after treatment with P7C3, with cerium oxide nanoparticles (CNPs), or with a combination of P7C3 and CNPs. FIG. 5A shows micrographs, both low and high magnification, of bone mineral formation when treatment was administered 24 hours after exposure to radiation and FIG. 5B shows micrographs, both low and high magnification, of bone formation when treatment was administered 48 hours after exposure to radiation (Alizarin red, 28 days).

FIGS. 5C-D show graphs quantifying new bone deposition after treatment with P7C3, with cerium oxide nanoparticles (CNPs, 64C), or with a combination of P7C3 and CNPs (64C-73) wherein treatment was administered 24 hours after exposure to radiation (post-radiation, Alizarin red). FIG. 5C shows a graph quantifying new bone deposition at 14 days post-radiation and FIG. 5D shows a graph quantifying new bone deposition at 28 days post-radiation. **** p<0.0001

FIGS. 5E-F show graphs quantifying new bone deposition after treatment with P7C3, with cerium oxide nanoparticles (CNPs, 64C), or with a combination of P7C3 and CNPs (64C-73) wherein treatment was administered 48 hours after exposure to radiation (post-radiation, Alizarin red). FIG. 5E shows a graph quantifying new bone deposition at 14 days post-radiation and FIG. 5F shows a graph quantifying new bone deposition at 28 days post-radiation. **** p<0.0001

FIGS. 6A-B show results of an alkaline phosphatase (ALP) assay. The micrographs show assessed bone formation after treatment with P7C3, with cerium oxide nanoparticles (CNPs), or with a combination of P7C3 and CNPs. FIG. 6A shows micrographs, both low and high magnification, of bone formation when treatment was administered 24 hours after exposure to radiation and FIG. 6B shows micrographs, both low and high magnification, of bone formation when treatment was administered 48 hours after exposure to radiation.

FIGS. 7A-B are graphs showing data resulting from XTT assay of macrophages. The XTT assay is used to quantify cell metabolic activity, which is a measure of cell viability, proliferation, and cytotoxicity. FIG. 7A shows the graph results of macrophages cultured for 3 days and FIG. 7B shows the graph results of macrophages cultured for 6 days.

FIGS. 8A-D are graphs showing gene expression of pro-inflammatory markers after treatment with P7C3, with cerium oxide nanoparticles (CNPs, 64C), or with a combination of P7C3 and CNPs (64C-73) wherein treatment was administered 24 hours after exposure to radiation (post-radiation treatment). FIG. 8A shows gene expression of interleukin-1β (II-1β); FIG. 8B shows gene expression of interleukin-6 (IL-6); FIG. 8C shows gene expression of receptor activator of nuclear factor kappa-B ligand (RANKL); and FIG. 8D shows gene expression of cathepsin-K (CTSK). **** p<0.0001; *** p<0.001; ** p<0.01

FIG. 9 shows micrographs of assessed DNA damage after treatment with P7C3, with cerium oxide nanoparticles (CNPs, 64C), or with a combination of P7C3 and CNPs (64C-73). The left panel shows micrographs, both low and high magnification, of DNA damage when treatment was administered 24 hours after exposure to harmful levels of radiation (7 Gy) and the right panel shows micrographs, both low and high magnification, of a control group which was not exposed to radiation. The green “comet” indicates DNA damage (red arrows).

FIGS. 10A-B are graphs showing quantification of DNA damage after treatment with P7C3, with cerium oxide nanoparticles (CNPs, 64C), or with a combination of P7C3 and CNPs (64C-73). FIG. 10A shows tail length (μm) and FIG. 10B shows comet length (μm). **** p<0.0001

### DETAILED DESCRIPTION OF THE INVENTION

As required, embodiments are disclosed herein; however, it is to be understood that the disclosed embodiments are merely examples and that the methods described below can be embodied in various forms. Therefore, specific structural and functional details disclosed herein are not to be interpreted as limiting, but merely as a representative basis for teaching one skilled in the art to variously employ the present subject matter in virtually any appropriately detailed structure and function. Further, the terms and phrases used herein are not intended to be limiting, but rather, to provide an understandable description of the concepts.

It can be advantageous to set forth definitions of certain words and phrases used throughout this disclosure. The terms “a” or “an”, as used herein, are employed to describe elements and components described herein. This is done merely for convenience and to give a general sense of the scope of the disclosure. This description should be read to include one or at least one and the singular also includes the plural unless it is obvious that it is meant otherwise. The term plurality, as used herein, is defined as two or more than two. The term another, as used herein, is defined as at least a second or more.

The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, or C; A and B; A and C; B and C; and A, B, and C.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Note that not all of the activities described above in the general description, or the examples are required, that a portion of a specific activity may not be required, and that one or more further activities can be performed in addition to those described. Still further, the order in which activities are listed is not necessarily the order in which they are performed.

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described compositions, nanocompositions, nanoparticles, nanozymes, pharmaceutical compositions, kits, and/or methods along with any further application of the principles of the invention as described herein, are contemplated as would normally occur to one skilled in the art to which the invention relates.

### Introduction to Experiments

A chimeric molecule is an engineered construct in which two or more components are linked to form a novel biological agent. As such, a novel aspect of the described invention is in combining multifunctional P7C3 to multifunctional cerium oxide nanoparticles (CNPs) for use as a double-acting medical strategy for counteracting bone tissue damage due to exposure to radiation and improve quality of life for patients while simultaneously inhibiting cancer growth.

Cleavable crosslinkers can be used to attach/link the small molecule to the nanoparticle, such that both ‘components’ are subsequently separated from one another following immediate exposure to body fluids. Nonlimiting examples of such cleavable crosslinkers include CD1 (N,N′-carbonyldiimidazole) and polyethyleneimine (commercially available from Sigma-Aldrich, ThermoFisher Scientific). Separation of the components occurs due to factors within the body breaking the crosslinks, leaving the P7C3 and nanoparticles to each work independently, and systemically via their unique mechanisms. Both the P7C3 and cerium oxide nanoparticles (CNPs) exert distinct pharmacological activity, thereby delivering a synergistic bifunctional effect.

### Generation of Cerium Nanozymes¹⁹

The properties that rare earth metals exhibit make them a remarkable strategic resource. The chemistry of rare earth metals differs from other main group metals because of the nature and occupation of the 4f orbitals, which in turn, imparts unique catalytic, magnetic, and electronic properties²⁰. These unusual properties can be exploited to create new technologies that are not possible with transition and main group metals. Unique nanozymes such as cerium oxide nanoparticles (CeONPs) are a new generation of artificial enzymes that have received much attention because of their exemplary nanozymatic activities, low toxicity, and ability to easily and drastically adjust their electronic configurations in response to changes in the bioenvironment^{21,22}. These properties are derived from quick and expedient interconversion of the oxidation state between Ce⁴⁺ (fully oxidized) and Ce³⁺ (fully reduced). The CeONPs feature oxygen vacancies or defects in the lattice structure, which arise through loss of oxygen and/or electrons when altering between CeO₂ and CeO₂₋ₓ during redox reactions. Through this dual and regenerative role as an oxidation and reduction catalyst many studies have shown that CeONPs possess multiple antioxidant-enzyme-like activities, including SOD (superoxide dismutase), CAT (catalase), and peroxidase-like activities. As such, they are expected to scavenge almost all types of noxious reactive species, under suitable conditions, to outperform endogenous antioxidants^{23,24,20,25}.

Synthesis of Nanozymes, CeONP^{60/40} and CeONP^{20/80}

The low formation energies of surface oxygen vacancies are important for oxidation, and the localization of charge as Ce³⁺ state provides power for reduction, altering the electronic configuration, catalytic properties, and response to ROS (reactive oxygen species) 26,27. The relative fractions of Ce³⁺ and Ce⁴⁺ surface sites (CeONPs^{3+/4+}) were adjusted to form two particle formulations (CeONP^{60/40}, rich in reduced state cerium sites and CeONP^{20/80}, reduced state lean) thereby altering the electronic configuration, catalytic properties, and response to ROS.

### Synthesis of a Nanozyme Designed for Greater Relative SOD (Superoxide Dismutase) Activity (CeONP^{60/40})

Synthesis was performed based on a previously published protocol²⁶. Cerium (III) nitrate hexahydrate (99.999% purity; Sigma Aldrich) was added to 50 mL of deionized water and allowed to dissolve completely. The Ce³⁺ ions were converted to a highly hydrated, cerium (IV) oxide through addition of 3% hydrogen peroxide to a pH below 3.5 and a final cerium concentration of 5 mM. From here, the solutions were left standing away from light and aged for up to 8 weeks. Aging was performed to allow degradation of excess hydrogen peroxide by catalytic surface reactions and for equilibration of particle phase composition/surface character (aging effects).

### Synthesis of a Nanozyme Designed for Greater Relative CAT (Catalase) Activity (CeONP^{20/80})

A forced hydrolysis technique was used to generate nanoparticles tuned to possess a lower fraction of Ce³⁺ surface sites, relative to Ce⁴⁺. Synthesis was performed based on a previously published protocol²⁷. Specifically, 1.24 g of cerium (III) nitrate hexahydrate (99.999% purity; Sigma Aldrich) was stirred in 50 mL of water for 1 hour followed by titration with 30% ammonium hydroxide (ACS grade, Alfa Aesar) to force precipitation of nanocrystalline cerium (hydro-) oxide over 4 hours of stirring. The solution was then centrifuged at 10,000 rpm to collect the sedimented particles and the sediment was washed 3 times with deionized water (to purify/isolate nanomaterial products and promote oxidation to cerium oxide). Particles were then resuspended in fresh deionized water and ultra-sonicated for 20 minutes to disperse stable particles. Solutions were left standing overnight and any further sediment was removed by manually collecting supernatant of well-suspended particles.

### Combination of P7C3 and Cerium Oxide Nanoparticles (CNPs) in Radiation Exposure

Macrophages, osteoclasts, stem cells, fat cells, and bone cells must all work together beneficially for bone to form in the body.

### Macrophages

Macrophages control inflammation and directly regulate the function or

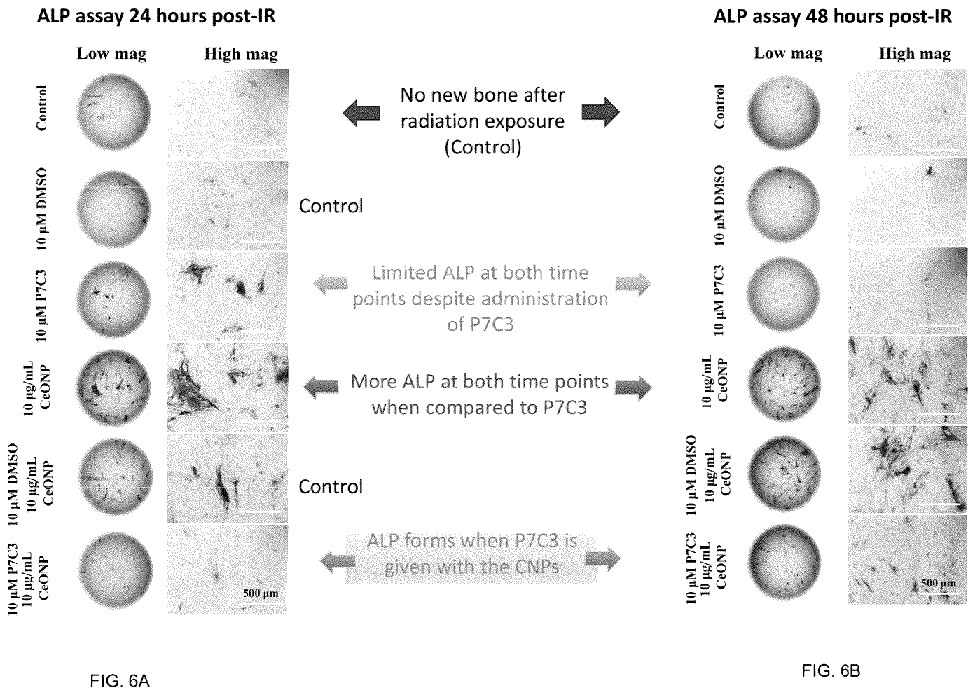

dysfunction of tissues, including bone tissue. Macrophages were treated with P7C3 alone, cerium oxide nanoparticles (CNPs) alone, or with a combination of P7C3 and CNPs (FIGS. 7A-B). No toxicity was shown.

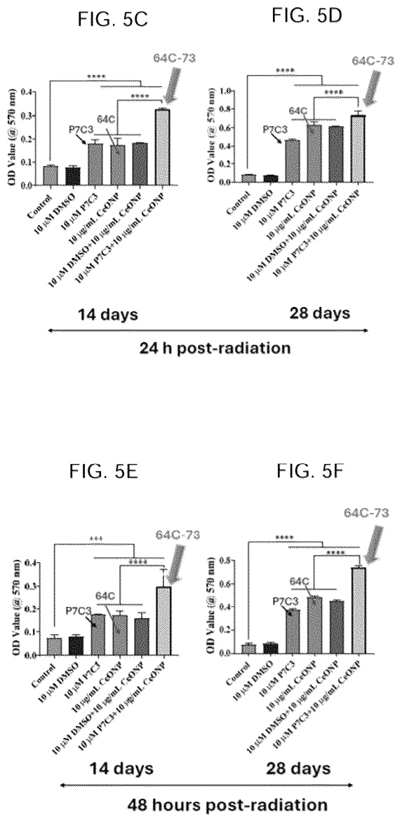

Specifically, macrophages were treated and cultured for 3 days (FIG. 7A) and 6 days (FIG. 7B) in the presence of CNPs, P7C3, or P7C3+CNPs. An XTT assay was used to quantify cell metabolic activity, which is a measure of cell viability, proliferation, and cytotoxicity. When compared with untreated control cells, no reduction in activity was found when cells were supplemented with P7C3 (yellow arrow), CNPs (blue arrow), or both P7C3+CNPs (green arrow), showing that treatment either individually or in combination delivered no toxicity to macrophages. Treatment was carried out both with and without exposure to radiation.

### Osteoclasts

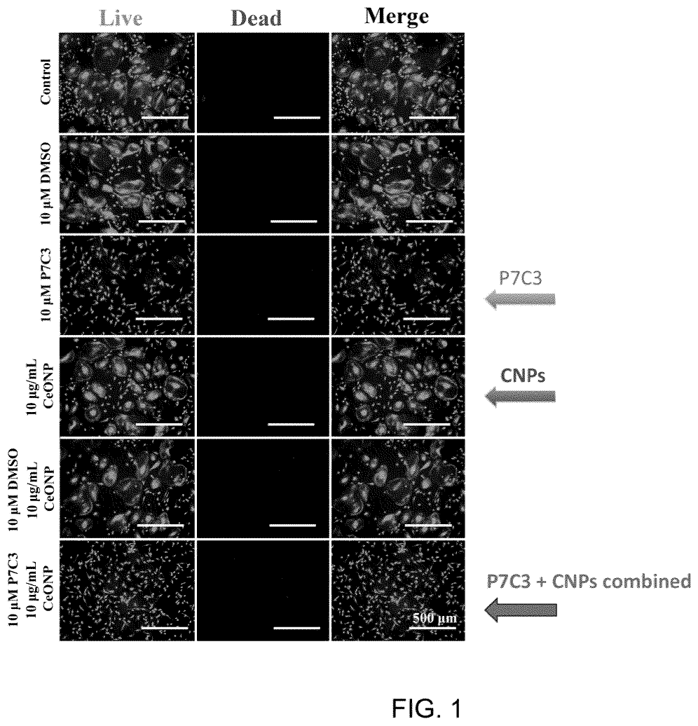

Osteoclasts are bone resorbing cells. When osteoclasts mature and are overactive, bone structure and mass is lost. Osteoclasts were treated with P7C3, with cerium oxide nanoparticles (CNPs), or with a combination of P7C3 and CNPs. These experiments were carried out without exposing the osteoclasts to radiation. FIG. 1 shows the results of the cell viability analysis. Green color indicates live cells and red color indicates dead cells. No upregulation in the number of red cells is seen after treatment. Thus, these results provide evidence that P7C3, CNPs, and their combination are non-toxic to the osteoclasts.

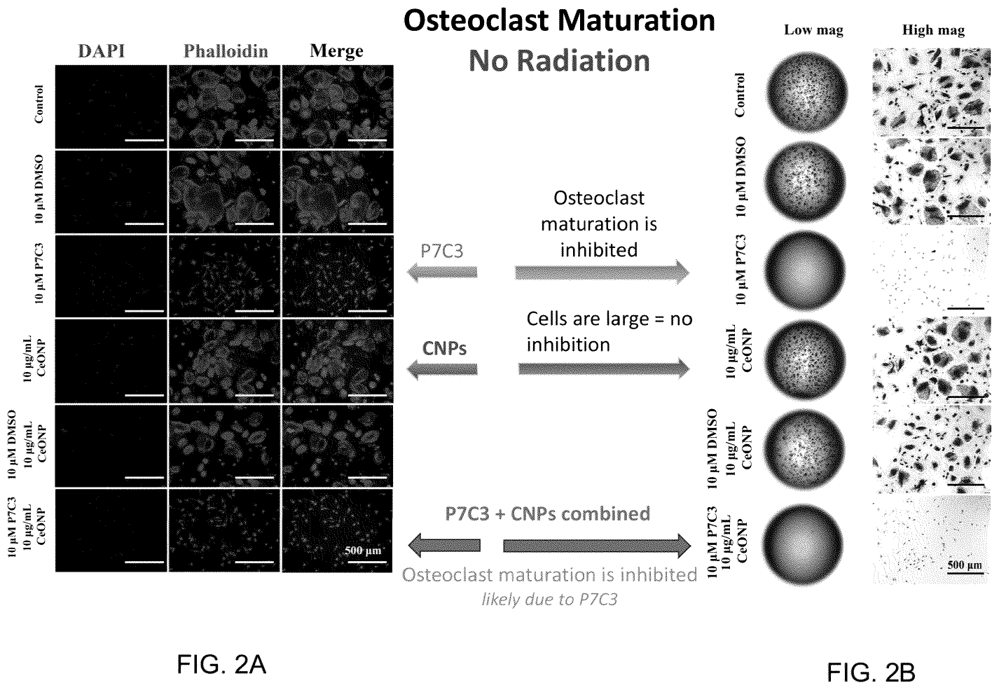

Additionally, as shown in FIGS. 2A-B, P7C3 stops the maturation of osteoclasts and therefore also stops the resorption activity of these cells. CNPs had no effect.

### Stem Cells and Fat Formation

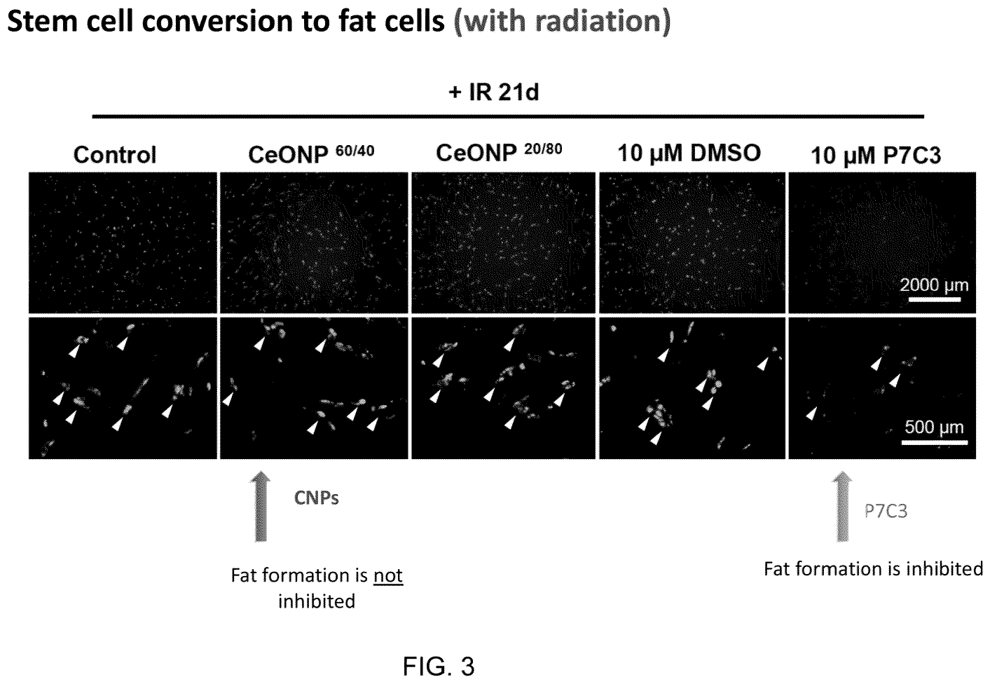

Radiation exposure, post-menopausal osteoporosis, and diabetes are conditions that cause stem cells to favor conversion to fat cells instead of conversion to bone cells. This process progresses to tissue dysfunction. Stem cells were treated with P7C3 or with cerium oxide nanoparticles (CNPs) after exposure to radiation. FIG. 3 shows this analysis of stem cell conversion to fat cells after treatment with P7C3, with CeONP^{60/40}, or with CeONP^{20/80}. All treatment was administered with radiation (IR 21d). The results show that P7C3, but not CNPs, inhibit fat formation from stem cells after exposure to radiation.

### Stem Cells and Bone Formation

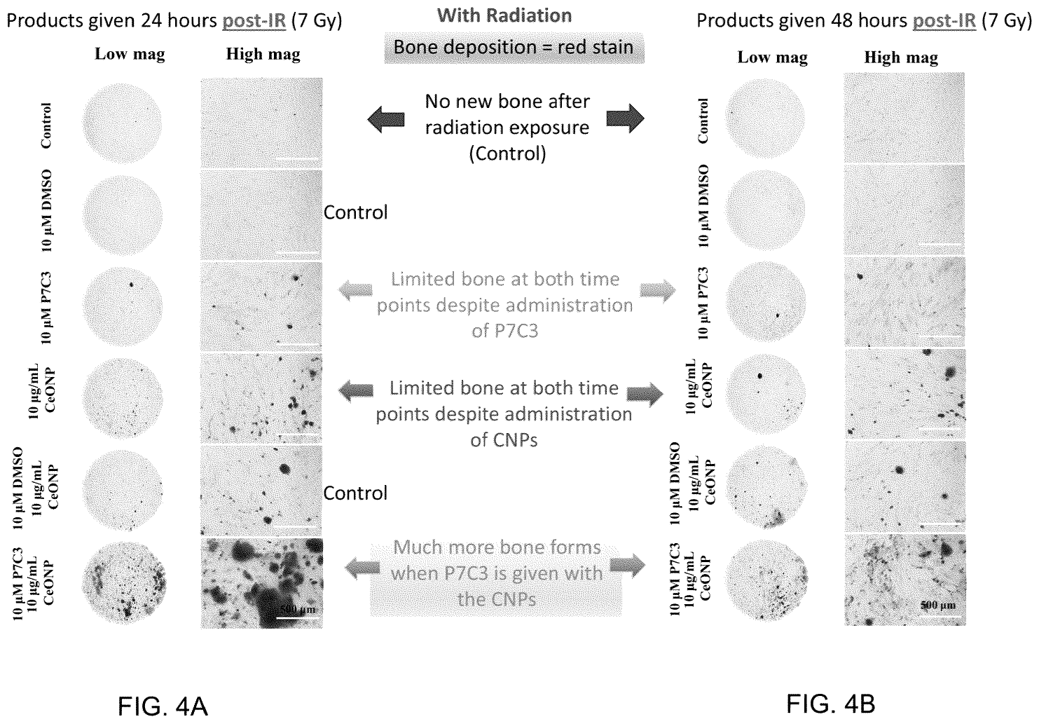

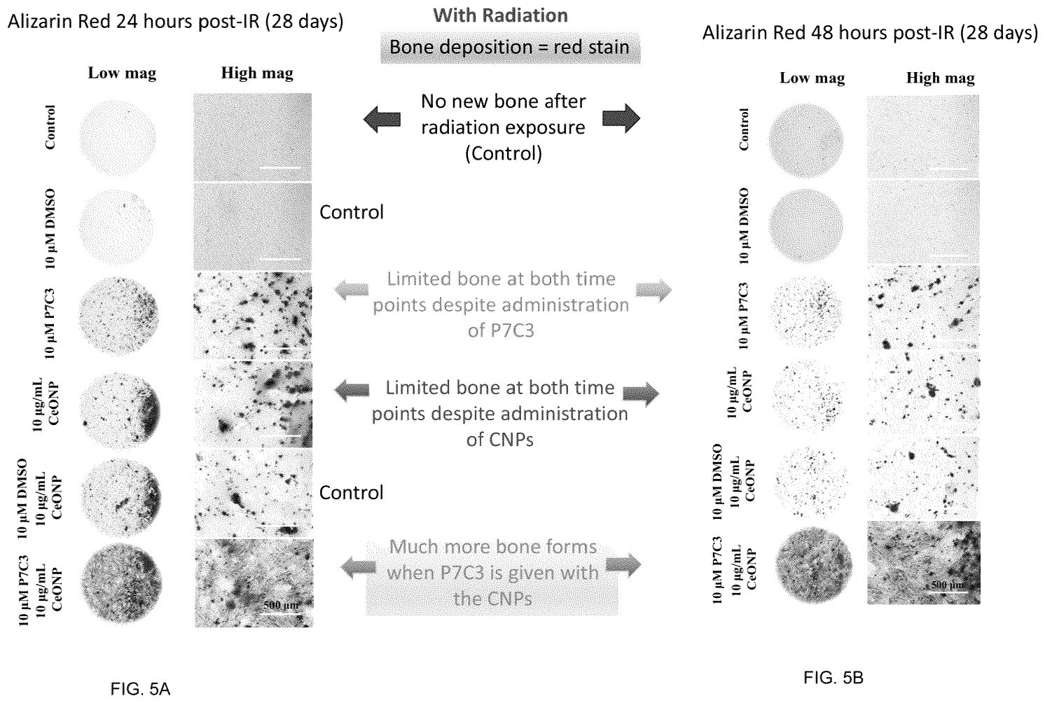

Radiation reduces the ability of bone cells to produce bone. Stem cells were treated with P7C3 or with cerium oxide nanoparticles (CNPs) after exposure to radiation (24 hours or 48 hours post-exposure). FIGS. 4A-B show micrographs of assessed bone formation after treatment with P7C3, with cerium oxide nanoparticles (CNPs), or with a combination of P7C3 and CNPs. FIG. 4A shows micrographs, both low and high magnification, of bone formation after 14 days of culture and when treatment was administered 24 hours after exposure to radiation and FIG. 4B shows micrographs, both low and high magnification, of bone mineral formation when treatment was administered 48 hours after exposure to radiation. The results show that administering P7C3 or CNPs separately to the stem cells does not substantially increase new bone deposition after 14 days. However, administering both P7C3 and CNPs together does increase bone deposition. This is further confirmed when the cells are cultured over a longer period of 28 days. FIG. 5A shows micrographs of now increased amounts of bone mineral formation when treatment was administered 24 hours after exposure, and FIG. 5B similarly confirms more bone deposition following treatment 48 hours after exposure. These results show more bone mineral deposition occurs over time, and that administering both P7C3 and CNPs continues to have a greater impact on enhancing bone deposition.

The data showing assessed bone formation was supported by quantification of new bone deposition. FIGS. 5C-D show graphs quantifying new bone deposition after treatment with P7C3, with cerium oxide nanoparticles (CNPs, 64C), or with a combination of P7C3 and CNPs (64C-73) wherein treatment was administered 24 hours after exposure to radiation (post-radiation, Alizarin red). FIG. 5C shows a graph quantifying new bone deposition at 14 days post-radiation and FIG. 5D shows a graph quantifying new bone deposition at 28 days post-radiation. (**** p<0.000) When treatment is administered 48 hours after radiation exposure, a statistically significant and a synergistic effect is seen where the amount of new bone deposited in the 64C-73 treatment group is much higher than either in the 64C group or in the P7C3 group.

FIGS. 5E-F show graphs quantifying new bone deposition after treatment with P7C3, with cerium oxide nanoparticles (CNPs, 64C), or with a combination of P7C3 and CNPs (64C-73) wherein treatment was administered 48 hours after exposure to radiation (post-radiation, Alizarin red). FIG. 5E shows a graph quantifying new bone deposition at 14 days post-radiation and FIG. 5F shows a graph quantifying new bone deposition at 28 days post-radiation. (**** p<0.000) The synergistic effect continues when treatment is administered more than 24 hours after radiation exposure. For example, when treatment is administered 48 hours after radiation exposure, a statistically significant and a synergistic effect is seen where the amount of new bone deposited in the 64C-73 treatment group is higher than either in the 64C group or in the P7C3 group.

The research shows that the mechanism through which the cerium oxide nanoparticles (CNPs) work is different from the mechanism of P7C3, as each affected the cells differently. It was anticipated that a combination of P7C3 and CNPs will produce an additive, synergistic effect. Currently, it is unknown why the combination of P7C3 and CNPs upregulates new bone formation. Without being bound by theory, it is hypothesized that since a mixture of several key proteins are required for bone formation to occur, P7C3 upregulates some of these proteins while CNPs upregulate others, thus resulting with the overall better effect. The current results suggest that CNPs upregulate the activity of the key bone proteins thereby boosting conversion of stem cells into bone forming cells, while P7C3 increases mineralization of the matrix that is deposited by the new bone forming cells. Thus, P7C3 and CNPs work on two different parts of the process that together boost new bone deposition.

The data of FIGS. 6A-B further highlights the different mechanisms of action of P7C3 and CNPs. FIGS. 6A-B show results of an alkaline phosphatase (ALP) assay. The micrographs show assessed bone formation after treatment with P7C3, with cerium oxide nanoparticles (CNPs), or with a combination of P7C3 and CNPs. FIG. 6A shows micrographs, both low and high magnification, of bone formation when treatment was administered 24 hours after exposure to radiation and FIG. 6B shows micrographs, both low and high magnification, of bone formation when treatment was administered 48 hours after exposure to radiation. The blue color indicates that mesenchymal stem cells are expressing more alkaline phosphatase and therefore are changing to osteoblasts, the bone depositing cells. It appears that CNPs predominantly drive this process following exposure to ionizing radiation (IR), while P7C3 appears to confer limited upregulation to these cells under these conditions. Thus, it appears that CNPs are more effective than P7C3 at converting stem cells to bone depositing cells.

Overall, the experimental results, as described and shown herein, indicate that administration of P7C3 and cerium oxide nanoparticles (CNPs) as a combination is non-toxic to cells and boosts bone formation after radiation exposure more so than when either P7C3 or CNPs is administered alone. Further, the inventive nanocompositions (64C-73) are more effective at regenerating cells after exposure to harmful levels of radiation than when either 64C (cerium oxide nanoparticles) or P7C3 are given alone.

### Pro-Inflammatory Markers

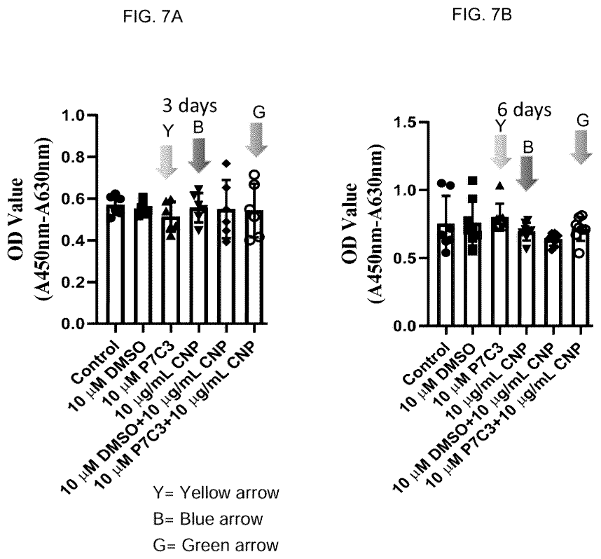

Radiation exposure increases levels of harmful, pro-inflammatory proteins such as interleukin-1β (Il-1β), interleukin-6 (IL-6), receptor activator of nuclear factor kappa-B ligand (RANKL), and cathepsin-K (CTSK). These proteins are associated with inducing inflammation, and ultimately, tissue damage. FIGS. 8A-D are graphs showing gene expression of pro-inflammatory markers after treatment with P7C3, with cerium oxide nanoparticles (CNPs, 64C), or with a combination of P7C3 and CNPs (64C-73) wherein treatment was administered 24 hours after exposure to radiation (post-radiation). FIG. 8A shows gene expression of interleukin-1β (II-1β); FIG. 8B shows gene expression of interleukin-6 (IL-6); FIG. 8C shows gene expression of receptor activator of nuclear factor kappa-B ligand (RANKL); and FIG. 8D shows gene expression of cathepsin-K (CTSK). (**** p<0.0001; *** p<0.001; ** p<0.01) A trend is seen where 64C-73 is more effective at lowering the gene expression of these protein markers compared to either 64C or P7C3 alone. A statistically significant decrease caused by 64C-73 is seen in the Il-1β and Il-6 groups compared to either 64C or P7C3 alone. Lower levels are also seen in the RANKL and CTSK groups in response to 64C-73.

### Post-Radiation-Induced DNA Damage

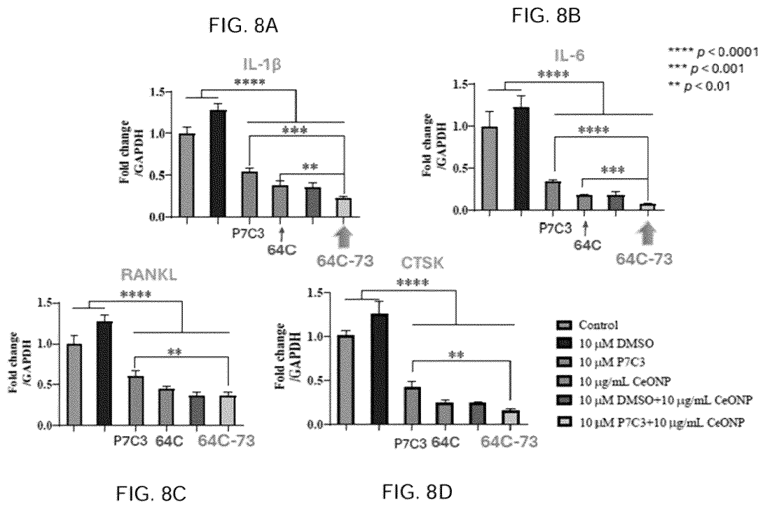

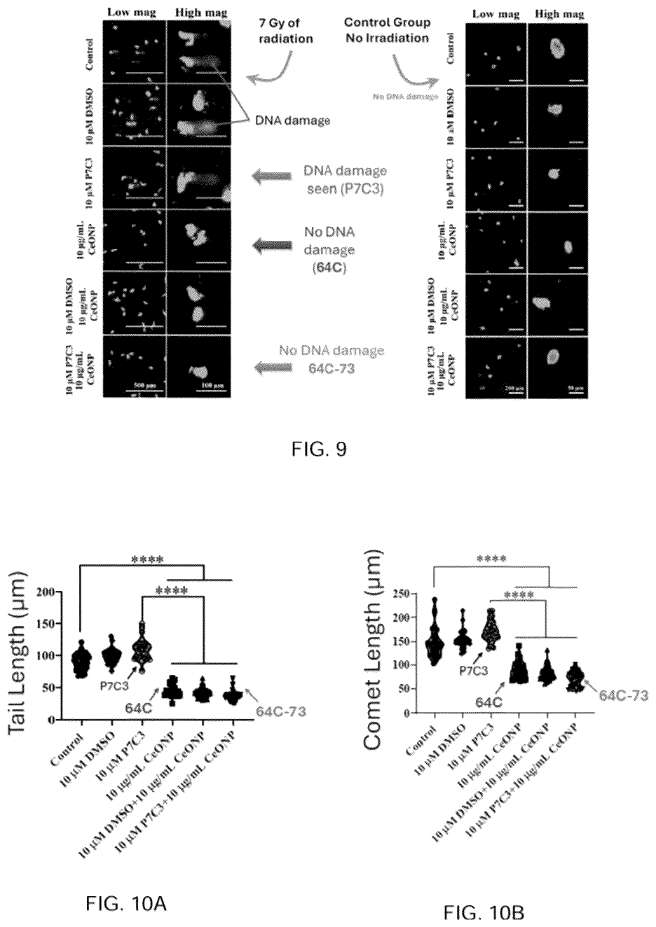

The inventive nanocompositions can protect cellular DNA from damage resulting from radiation exposure. In this assessment, 64C and 64C-73 were given 24 hours after exposure to harmful levels of harmful levels of radiation (7 Gy). Despite this radiation, no comets (no DNA damage) were seen when 64C and 64C-73 were given. The absence of comets is indicative of the protective effect. In contrast, when given alone, P7C3 is not shown to protect against DNA damage. FIG. 9 shows results of a comet assay. In this assay, as the frequency of DNA breaks (indicating damage) increases so does the fraction of DNA extending toward the anode during electrophoresis. This extension is referred to as a “comet” or “comet tail.” FIG. 9 shows micrographs of assessed DNA damage after treatment with P7C3, with cerium oxide nanoparticles (CNPs, 64C), or with a combination of P7C3 and CNPs (64C-73). The left panel shows micrographs, both low and high magnification, of DNA damage when treatment was administered 24 hours after exposure to harmful levels of radiation (7 Gy) and the right panel shows micrographs, both low and high magnification, of a control group which was not exposed to radiation. The green “comet” indicates DNA damage (red arrows). The quantification of the DNA damage is shown in FIGS. 10A-B. These graphs show quantification of DNA damage after treatment with P7C3, with cerium oxide nanoparticles (CNPs, 64C), or with a combination of P7C3 and CNPs (64C-73). FIG. 10A shows tail length (μm) and FIG. 10B shows comet length (μm). 64C and 64C-73 both significantly reduce DNA damage compared to P7C3 when administered alone. **** p<0.0001

### Combination of P7C3 and Cerium Oxide Nanoparticles (CNPs) in Osteoporosis

The above embodiments also relate to the field of medicine for an article of manufacture delivering a prophylactic and therapeutic tissue strategy for osteoporosis. The remarkable increase in average life expectancy during the 20th century ranks as one of society's greatest achievements. As a result, the world's elderly population (aged ≥65 years) continues to grow at an unprecedented rate. As the proportion of the oldest-old (aged >85 years) and length of life expectancy increases, a rise in age-related degenerating diseases, disability, and prolonged dependency is projected. Age-related bone loss and osteoporosis is inevitable in both men and women, and can lead to fragility fractures, immobility, and disability. Currently, bisphosphonate treatment and weight-bearing exercise are commonly used therapeutic approaches, but both with limited long-term success. The administration of a cerium oxide nanoparticle (CNPs) formulation with increased levels of Ce3+ surface sites relative to Ce4+, specifically upregulates new bone deposition. Further, and via a differing mechanism, P7C3, reduces the progression of post-menopausal osteoporosis in animals. It is shown that combination of P7C3 and CNPs will produce an additive, synergistic effect in the prevention and/or treatment of osteoporosis.

### CONCLUSION

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112 (f) with respect to any of the appended representative claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the representative claims.

After reading the disclosure, skilled artisans will appreciate that certain features are, for clarity, described herein in the context of separate embodiments can also be provided in combination in a single embodiment. Conversely, various features that are, for brevity, described in the context of a single embodiment, can also be provided separately or in any sub-combination. Further, references to values stated in ranges include each and every value within that range.

The above discussion is meant to be illustrative of the principles and various embodiments of the present invention. Numerous variations and modifications will become apparent to those skilled in the art once the above disclosure is fully appreciated. It is intended that the following representative claims be interpreted to embrace all such variations and modifications. Thus, the compositions, nanocompositions, nanoparticles, nanozymes, pharmaceutical compositions, kits, and/or methods described herein are presently representative of the preferred embodiments, are intended to be exemplary and are not intended as limitations on the scope.

### REFERENCES

1. Bayat Mokhtari, et al. Combination therapy in combating cancer. Oncotarget 8, 38022-38043 (2017).
2. Berkey, F. J. Managing the adverse effects of radiation therapy. Am. Fam. Physician 82, 381-388 (2010).
3. Jaffray, D. A. et al. Global task force on radiotherapy for cancer control. Lancet Oncol. 16, 1144-1146 (2015).
4. Jarosz-Bieg, M. et al. Tumor microenvironment as a “game changer” in cancer radiotherapy. Int. J. Mol. Sci. 20, 3212 (2019).
5. Curi, M. M. et al. Histopathologic and histomorphometric analysis of irradiation injury in bone and the surrounding soft tissues of the jaws. J. Oral. Maxillofac. Surg. 74, 190-199 (2016).
6. Daniel, M. et al. Overcoming nuclear winter: the cutting-edge science of bone healing and regeneration in irradiated fields. Plast. Reconstr. Surg. Glob. Open 9, e3605 (2021).
7. Oh, D. et al. Insufficiency fracture after radiation therapy. Radiat. Oncol. J. 32, 213 (2014).
8. Sparks, R. B. et al. Radiation dose distributions in normal tissue adjacent to tumors containing (131) 1 or (90) Y: the potential for toxicity. J. Nucl. Med. 43, 1110-1114 (2002).
9. Willey, J. S. et al. Ionizing radiation and bone loss: space exploration and clinical therapy applications. Clin. Rev. Bone Min. Metab. 9, 54-62 (2011).
10. Soares, C. B. G. et al. Pathological fracture after radiotherapy: systematic review of literature. Rev. Assoc. Med. Bras. 65, 902-908 (2019).
11. Donaubauer, A. J. et al. The influence of radiation on bone and bone cells-differential effects on osteoclasts and osteoblasts. Int. J. Mol. Sci. 21, 6377 (2020).
12. Kim. H. J. et al. Fractures of the sacrum after chemoradiation for rectal carcinoma: incidence, risk factors, and radiographic evaluation. Int. J. Radiat. Oncol. 84, 694-699 (2012).
13. Aoki, M. et al. Radiation-induced rib fracture after stereotactic body radiotherapy with a total dose of 54-56 Gy given in 9-7 fractions for patients with peripheral lung tumor: impact of maximum dose and fraction size. Radiat. Oncol. 10, 99 (2015).
14. Blomlie, V. et al. Incidence of radiation-induced insufficiency fractures of the female pelvis: evaluation with MR imaging. Am. J. Roentgenol. 167, 1205-1210 (1996).
15. Wei, F. et al. A novel multifunctional radioprotective strategy using P7C3 as a countermeasure against ionizing radiation-induced bone loss. Bone Research. 11:34 (2023).
16. Wang, G. et al. P7C3 neuroprotective chemicals function by activating the rate-limiting enzyme in NAD salvage. Cell. 158, 1324-1334 (2014).
17. Zhang, T. et al. Enzymes in the NAD⁺ salvage pathway regulate SIRT1 activity at target gene promoters. J. Biol. Chem. 284, 20408-20417 (2009).
18. Bauman, M. D. et al. Neuroprotective efficacy of P7C3 compounds in primate hippocampus. Transl. Psychiatry. 8, 202 (2018).
19. Wei, F. et al. A novel approach for the prevention of ionizing radiation-induced bone loss using a designer multifunctional cerium oxide nanozyme. Bioactive Materials. 21, 547-565 (2023).
20. Xu, C. et al. Cerium oxide nanoparticle: a remarkably versatile rare earth nanomaterial for biological applications, NPG Asia Mater. 6, (No. 3) (2014).
21. Celardo, I. et al. Pharmacological potential of cerium oxide nanoparticles. Nanoscale 3, (No. 4) (2011).
22. Yu, Z. et al. Nanoenzymes in disease diagnosis and therapy. Chem. Comm. 56, (No. 99) (2020).
23. Halliwell, B. et al. Hydroxyl radical is a significant player in oxidative DNA damage in vivo. Chem. Soc. Rev. 50, (No. 15) (2021).
24. Ighodaro, O. M. et al. First line defense antioxidants-superoxide dismutase (SOD), catalase (CAT), and glutathione peroxidase (GPX): their fundamental role in the entire antioxidant defense grid. Alexandria J. Med. 54, (No. 4) (2018).
25. Song, G. et al. Nanoscale cerium oxide: synthesis, biocatalytic mechanism, and applications. Catalyst 11, (No. 9) (2021).
26. Hirst, S. M. et al. Antiinflammatory properties of cerium oxide nanoparticles. Small 5, (No. 24) (2009).
27. Das, S. et al. The induction of angiogenesis by cerium oxide nanoparticles through the modulation of oxygen in intracellular environments. Biomaterials 33, (No. 31) (2012).

What is claimed is:

1. A pharmaceutical composition comprising a therapeutically effective amount of a nanocomposition including P7C3 (aminopropyl carbazole) attached to cerium oxide nanoparticles and at least one pharmaceutically acceptable carrier.

2. The pharmaceutical composition according to claim 1, wherein the cerium oxide nanoparticles have increased levels of Ce³⁺ surface sites relative to levels of Ce⁴⁺ surface sites.

3. The pharmaceutical composition according to claim 2, where the cerium oxide nanoparticles having increased levels of Ce³⁺ surface sites relative to levels of Ce⁴⁺ surface sites is cerium oxide CeONP^{60/40}.

4. The pharmaceutical composition according to claim 1, wherein the P7C3 is attached to the cerium oxide nanoparticles via a crosslinker.

5. The pharmaceutical composition according to claim 4, wherein the crosslinker is CD1 (N,N′-carbonyldiimidazole) or polyethyleneimine.

6. A method for enhancing bone deposition in a subject in need thereof, the method comprising:
providing a pharmaceutical composition including a nanocomposition having P7C3 (aminopropyl carbazole) attached to cerium oxide nanoparticles and at least one pharmaceutically acceptable carrier; and
administering the pharmaceutical composition to the subject.

7. The method according to claim 6, wherein the subject is a patient receiving radiation therapy, a patient that will receive radiation therapy, a patient that has received radiation therapy, a patient having osteoporosis, a patient having a condition that causes osteoporosis, a patient having cancer, or a patient having bone trauma/injury.

8. A method for protecting bone tissue of a subject from radiation damage, the method comprising:
providing a pharmaceutical composition including a nanocomposition having P7C3 (aminopropyl carbazole) attached to cerium oxide nanoparticles and at least one pharmaceutically acceptable carrier; and
administering the pharmaceutical composition to the subject.

9. The method according to claim 8, wherein the cerium oxide nanoparticles have increased levels of Ce³⁺ surface sites relative to levels of Ce⁴⁺ surface sites.

10. The method according to claim 8, wherein administering the pharmaceutical composition to the subject includes administering the pharmaceutical composition to the subject prior to radiation exposure, prior to radiation therapy, concurrently with radiation therapy, or at a predetermined time after radiation therapy.

11. The method according to claim 10, wherein the predetermined time after radiation therapy is 24 hours or 48 hours.

12. A method for protecting bone tissue from radiation damage in a subject undergoing radiation therapy, the method comprising:
providing the pharmaceutical composition according to claim 1; and
administering the pharmaceutical composition to the subject.

13. A method for treating cancer and protecting bone tissue from radiation damage in a subject undergoing radiation therapy for the cancer, the method comprising:
providing the pharmaceutical composition according to claim 1; and
administering the pharmaceutical composition to the subject.

14. The method according to claim 13, wherein administering the pharmaceutical composition to the subject includes administering the pharmaceutical composition to the subject prior to the radiation therapy, concurrently with the radiation therapy, or at a predetermined time after the radiation therapy.

15. A method for treating osteoporosis in a subject in need thereof, the method comprising:
providing the pharmaceutical composition according to claim 1; and
administering the pharmaceutical composition to the subject.

16. A method for treating bone trauma/injury in a subject in need thereof, the method comprising:
providing the pharmaceutical composition according to claim 1; and
administering the pharmaceutical composition to the subject.

17. A method for protecting DNA of bone cells exposed to radiation in a subject in need thereof, the method comprising:
providing the pharmaceutical composition according to claim 1; and
administering the pharmaceutical composition to the subject.

18. The method according to claim 17, wherein administering the pharmaceutical composition to the subject includes administering the pharmaceutical composition to the subject prior to radiation exposure, concurrently with radiation exposure, or at a predetermined time after radiation exposure.

19. A method for reducing gene expression of pro-inflammatory markers in a subject in need thereof, the method comprising:
providing the pharmaceutical composition according to claim 1; and
administering the pharmaceutical composition to the subject.

20. The method according to claim 19, wherein the pro-inflammatory markers are one or more of interleukin-1β (II-1β), interleukin-6 (IL-6), receptor activator of nuclear factor kappa-B ligand (RANKL), and cathepsin-K (CTSK).
